(19) United States
(12) Patent Application Publication
(10) Pub. No.: US 2019/0177749 A1
(43) Pub. Date: Jun. 13, 2019

(54) PROCESS AND SYSTEM FOR SEPARATION OF A STARCH RICH FLOW

(71) Applicant: CELLULOSIC ETHANOL TECHNOLOGIES, LLC, Galva, IA (US)

(72) Inventor: Travis Brotherson, Holstein, IA (US)

(21) Appl. No.: 16/307,770

(22) PCT Filed: Jun. 20, 2017
(86) PCT No.: PCT/US17/38239

§ 371 Date: Dec. 6, 2018

Related U.S. Application Data
(60) Provisional application No. 62/352,826, filed on Jun. 21, 2016.

(51) Int. Cl. C12P 7/10; C12P 7/16

(52) U.S. Cl. CPC C12P 7/10; C12P 7/16

(57) ABSTRACT

The invention is directed to a method of producing a biomass-derived product, comprising filtering through at least one paddle screen a high solids liquefaction slurry that comprises starch and fiber, thereby separating the starch into a starch rich stream and the fiber into a fiber rich stream; and fermenting the starch rich stream to produce a biomass-derived product.

### FIELD OF THE INVENTION

The invention relates to a process for producing and utilizing a starch rich flow when processing plant biomass.

### BACKGROUND OF THE INVENTION

The production of ethanol for use as a gasoline additive or a straight liquid fuel continues to increase as petroleum costs rise and environmental concerns become more pronounced. Ethanol is generally produced using conventional fermentation processes that convert the starch in plant-based feedstocks into ethanol. However, the yeasts in these conventional fermentation processes are only able to convert limited concentrations of starch in these feedstocks and, therefore, can leave fermentable starch and other valuable sugars in the fermentation byproducts. Consequently, this can result in a reduced yield of ethanol from a bushel of grain and, ultimately, high concentrations of valuable starch leaving the bioprocessing plant in the fermentation byproducts. Thus, there is a need for a process and system that can maximize the potential of all the starch present in fermentation feedstocks.

The present invention overcomes previous shortcomings in the art by providing a process for producing and utilizing a starch rich flow when processing biomass.

### SUMMARY OF THE INVENTION

One aspect of the invention provides a method of producing a biomass-derived product, comprising: filtering through at least one paddle screen a high solids liquefaction slurry (i.e., a high solids liquifact) that comprises starch and fiber, thereby separating the starch into a starch rich stream and the fiber into a fiber rich stream; and fermenting the starch rich stream to produce a biomass-derived product.

A second aspect provides a method of processing a high solids liquefaction slurry to produce a biomass-derived product, comprising: filtering through at least one paddle screen a high solids liquefaction slurry that comprises starch and fiber, thereby separating the starch into a starch rich stream and the fiber into a fiber rich stream; and fermenting the starch rich stream to produce a biomass-derived product.

Further provided are products produced from the methods of the invention.

These and other aspects of the invention are set forth in more detail in the description of the invention below.

### BRIEF DESCRIPTION OF THE DRAWINGS

FIG. 1 provides a schematic of the bioprocessing system comprising at least one screen paddle.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like refers to variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the tetra “consisting essentially of” when used in a claim of this invention is not intended to be interpreted to be equivalent to “comprising.”

As used herein, the term “increase,” “increasing,” “increased,” “enhance,” “enhanced,” “enhancing,” and “enhancement” (and grammatical variations thereof) describe an elevation of at least about 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

The term “starch-digesting enzyme” includes any enzyme that can catalyze the transformation of a starch molecule or a degradation product of a starch molecule. For example, starch-digesting enzymes include starch-degrading or isomerizing enzymes including, for example, α-amylase (EC 3.2.1.1), endo or exo-1,4- or 1,6-α-D-glucoamylase, glucose isomerase, β-amylases (EC 3.2.1.2), α-glucosidases (EC 3.2.1.20), and other exo-amylases; starch debranching enzymes, such as isoamylase (EC 3.2.1.68), pullulanase (EC 3.2.1.41), neo-pullulanase, iso-pullulanase, amylopullulanase and the like; glycosyl transferases such as cyclodextrin glycosyltransferase and the like. Starch-digesting enzymes can be used in conjunction with other enzymes that can facilitate the release of starch from plant tissue. Starch-digesting enzymes can be used in conjunction with cellulases such as exo-1,4-β-cellobiohydrolase (EC 3.2.1.91), exo-1,3-β-D-glucanase (EC 3.2.1.39), hemicellulase, β-glucosidase and the like; endoglucanases such as endo-1,3-β-glucanase (EC 3.2.1.6) and endo-1,4-β-glucanase (EC 3.2.1.4) and the like; L-arabinases, such as endo-1,5-α-L-arabinase (EC EC 3.2.1.99), α-arabinosidases (EC 3.2.1.55) and the like; galactanases such as endo-1,4-β-D-galactanase (EC 3.2.1.89), endo-1,3-β-D-galactanase (EC 3.2.1.90), 1-galactosidase, α-galactosidase and the like; mannanases, such as endo-1,4-β-D-mannanase (EC 3.2.1.78), β-mannosidase (EC 3.2.1.25), α-mannosidase (EC 3.2.1.24) and the like; xylanases, such as endo-1,4-1-xylanase (EC 3.2.1.8), β-D-xylosidase (EC 3.2.1.37), 1,3-β-D-xylanase, and the like; pectinases and phytases. In some embodiments, the starch-digesting enzyme is α-amylase, pullulanase, α-glucosidase, glucoamylase, amylopullulanase, glucose isomerase, or combinations thereof.

The starch-digesting enzyme can be specifically selected based on the desired starch-derived end product, the end product having various chain lengths based on, e.g., a function of the extent of processing or with various branching patterns desired. For example, an α-amylase, glucoamylase, or amylopullulanase can be used under short incubation times to produce dextrin products and under longer incubation times to produce shorter chain products or sugars. A pullulanase can be used to specifically hydrolyze branch points in the starch yielding a high-amylose starch, or a neopullulanase can be used to produce starch with stretches of α-1,4 linkages with interspersed α-1,6 linkages. Glucosidases can be used to produce limit dextrins, or a combination of different enzymes can be used to make other starch derivatives. In some embodiments, a glucose-isomerase can be selected to convert the glucose (hexose) into fructose.

In particular, α-amylase refers to an enzyme which cleaves or hydrolyzes internal a (1-4) glycosidic bonds in starch to produce a 1-2 bonds and resulting in smaller molecular weight maltodextrins. These smaller molecular weight maltodextrins include, but are not limited to, maltose, which is a disaccharide (i.e., a dextrin with a degree of polymerization of 2 or a DP2), maltotriose (a DP3), maltotetrose (a DP4), and other oligosaccharides. The enzyme α-amylase (EC 3.2.1.1) can also be referred to as 1,4-α-D-glucan glucanohydrolase or glycogenase. A variety of α-amylases are known in the art and are commercially available. An α-amylase can be from a fungal or bacterial origin and can be expressed in transgenic plants. The α-amylase can be thermostable.

Glucoamylase (also known as amyloglucosidase) refers to the enzyme that has the systematic name 1,4-α-D-glucan glucohydrolase (E.C. 3.2.1.3). Glucoamylase removes successive glucose units from the non-reducing ends of starch. A variety of glucoamylases are known in the art and are commercially available. For example, certain glucoamylases can hydrolyze both the linear and branched giucosidic linkages of starch, amylose, and amylopectin. Glucoamylase can be from a fungal origin and can be expressed in transgenic plants. The glucoamylase can be thermostable.

The term “slurry” refers to a mixture of starch or a starch-containing material (e.g., milled corn) and an aqueous component, which can include, for example, water, de-ionized water, or a process water (i.e., backset, steam, condensate), or any combination thereof. The ter ins slurry and mash can be used interchangeably.

As used herein the terms “liquefaction,” “liquefy,” “liquefact,” and variations thereof refer to the process or product of converting starch to soluble dextrinized substrates (e.g., smaller polysaccharides). Liquefact can also be referred to as “mash.”

The term “secondary liquefaction” refers to a liquefaction process that takes place after an initial period of liquefaction or after a jet cooking step of a multi-stage liquefaction process. The secondary liquefaction can involve a different temperature than a previous liquefaction step or can involve the addition of additional starch-digesting enzymes (e.g., α-amylase).

As used herein, the terms “saccharification” and “saccharifying” refer to the process of converting polysaccharides to dextrose monomers using enzymes. Saccharification can specifically refer to the conversion of polysaccharides in a liquefact. Saccharification products are, for example, glucose and other small (low molecular weight) oligosaccharides such as disaccharides (a DP2) and trisaccharides (a DP3).

“Fermentation” or “fermenting” refer to the process of transforming sugars from reduced plant material to produce alcohols (e.g., ethanol, methanol, butanol, propanol); organic acids (e.g., citric acid, acetic acid, itaconic acid, lactic acid, gluconic acid, propionate); ketones (e.g., acetone), amino acids (e.g., glutamic acid); gases (e.g., H.sub.2 and CO.sub.2), antibiotics (e.g., penicillin and tetracycline); enzymes; vitamins (e.g., riboflavin, B.sub.12, beta-carotene); and/or hormones. Fermentation can include fermentations used in the consumable alcohol industry (e.g., beer and wine), dairy industry (e.g., fermented dairy products), leather industry, and tobacco industry. Thus, fermentation includes alcohol fermentation. Fermentation also includes anaerobic fermentations.

Fermenting can be accomplished by any organism suitable for use in a desired fermentation step. Suitable fermenting organisms are those that can convert DP1-3 sugars, especially glucose and maltose directly or indirectly to the desired fermentation product (e.g., ethanol, propanol, butanol or organic acid). Fermenting can be effected by a microorganism, such as fungal organisms (e.g., yeast or filamentous fungi). The yeast can include strains from a Pichia or Saccharomyces species. In some embodiments, the yeasts can include, but are not limited to, Saccharomyces cerevisiae, Pichia stipitis, Candida shehatae, and any combination thereof. In representative embodiments, the yeast can be Saccharomyces cerevisiae. In further embodiments, the yeast can be S. cerevisiae, P. stipites and C. shehatae.

Bacterial can also be used in a fermentation process. Bacteria can include but are not limited to species from Acetobacter, engineered E. coli, Clostridium, Acidophilus or Lactobacter.

Fermenting can include contacting a mixture including sugars from the reduced plant material with yeast under conditions suitable for growth of the yeast and production of ethanol. In some embodiments, fermenting involves simultaneous saccharification and fermentation (SSF). The amount of yeast employed can be selected to effectively produce a desired amount of ethanol in a suitable time.

“Slurry tank” refers to any tank used to contain ground plant material combined with a liquid. A commercial slurry tank is a slurry tank used in a commercial production setting which may be a dry grind ethanol plant, a grain milling plant using a wet or dry milling process to mill corn grain or may be a food production plant that is combining ground plant flour with liquids in order to form a slurry or liquefaction.

The term “hydrolysis” is defined as a chemical reaction or process in which a chemical compound is broken down by reaction with water. The starch digesting enzymes hydrolyze starch into smaller units as previously described.

Fermentation tank and fermentor refer to instruments that are used to ferment a substance to form alcohol. Dry grind ethanol plants may have several fermentation tanks which are used to produce ethanol from mash; however, any structure that allows fermentation to occur can be used with this invention.

Any starch source may be used with this invention. Plant material is often used as sources for starch. As used herein, the phrase “plant material” refers to all or part of any plant that includes starch. The plant material includes, but is not limited to, a grain, fruit, seed, stalk, wood, vegetable, or root. The plant material can be obtained from any plant including, but not limited to, sorghum (milo), oats, barley, wheat, berry, grape, rye, maize (corn), rice, potato, sugar beet, sugarcane, pineapple, yams, plantain, banana, grasses or trees. Suitable plant material includes grains such as maize (corn, e.g., whole ground corn), sorghum (milo), barley, wheat, rye, rice, and millet; and starchy root crops, tubers, or roots such as potato, sweet potato, and cassava. The plant material can also be obtained as a previously treated plant product such as soy cake generated during the processing of soybeans. The plant material can be a mixture of such materials and by-products of such materials, e.g., corn fiber, corn cobs, stover, or other cellulose- and hemicellulose-containing materials, such as wood or plant residues. Suitable plant materials can include, for example, corn, either standard corn or waxy corn.

Plant material can be processed by a variety of milling methods including but not limited to wet milling, dry milling, dry grinding, cracking, coarse grinding, fine grinding, fractionating, mixing, flaking, steam flaking, rolling or chopping. The corn wet milling process separates corn into its four basic components: starch, germ, fiber and protein. Typically, to accomplish this process, the incoming corn is first inspected and cleaned. Then it is steeped for approximately 30 to 40 hours to begin hydrolyzing the starch and breaking the protein bonds. Next, the process involves a coarse grind to separate the germ from the rest of the kernel. The remaining slurry that consists of fiber, starch and protein is finely ground and screened to separate the fiber from the starch and protein. The starch is separated from the remaining slurry. The starch then can be converted to syrup or it can be made into several other products through a fermentation process.

In dry milling, the corn is combined with water in a brief tempering process prior to grinding the corn to a flour. The ground corn flour is then fractionated into bran, germ and grits (starchy fractions). The starchy fractions from the dry milling process are used in the production of snack foods and other products including industrial products. The starchy fractions obtained from the dry milling process are typically not used in the production of ethanol.

In dry grinding, the entire corn kernel or other starchy grain is first ground into flour, which is referred to in the industry as “meal” and processed without separating out the various component parts of the grain. The meal is mixed with water or backset to form a “mash”. Enzymes are added to the mash to convert the starch to dextrose, a simple sugar. Ammonia is added for pH control and as a nutrient to the yeast

Embodiments of the invention comprising a method of producing and processing a starch rich flow can be incorporated into a wet milling, dry milling or dry grinding process.

To produce ethanol, starch containing fractions derived from wet milling or ground grain from dry grinding are further hydrolyzed into fermentable sugars which are then fermented to make ethanol. Several plant starch processing methods exist including a raw starch process, which involves little to no heating of the milled plant material being processed; or higher temperature hydrolysis of starch frequently referred to as “liquefaction”. In either of these methods for breaking down starch derived from plants, the conventional process involves the addition of enzymes, frequently liquid enzymes, to the milled plant starch in a slurry tank.

Liquefaction involves a starch gelatinization process, wherein aqueous starch slurry is heated so that the granular starch in the slurry swells and bursts, dispersing starch molecules into the solution. During the gelatinization process, there is a dramatic increase in viscosity. To enable handling during the remaining process steps, the starch must be thinned or “liquefied”. This reduction in viscosity can be accomplished by enzymatic degradation in a process referred to as liquefaction. During liquefaction, the long-chained starch molecules are degraded (hydrolyzed) into smaller branched and linear chains of glucose units (dextrins) by an enzyme, such as alpha-amylase (i.e., α-amylase). Starch-digesting enzymes can be added to the starch hydrolysis process as either liquid enzyme added when the milled plant material is mixed with water or can be delivered by using transgenic grain expressing the starch-digesting enzyme as described in U.S. Pat. No. 7,102,057. In some embodiments, the starch-digesting enzyme can be an alpha-amylase.

A conventional enzymatic liquefaction process comprises a three-step hot slurry process. The slurry is heated to between 80-85° C. to initiate gelatinization and α-amylase is added to initiate liquefaction. The slurry is jet-cooked at temperatures between 105 and 125° C. to complete gelatinization of the slurry, cooled to 60-95° C. (e.g., between 90-95° C.), and, usually, additional α-amylase is added to finalize hydrolysis during a secondary liquefaction step. This three step process is employed in order to break down as much of the plant starch as possible.

Liquefaction results in the generation of dextrins as the starch is hydrolyzed. The dextrins can be broken down further during saccharification, to produce low molecular weight sugars that can be metabolized by yeast. The saccharification hydrolysis is typically accomplished using glucoamylases and/or other enzymes such as α-glucosidases and/or acid α-amylases. A full saccharification step typically lasts up to 72 hours. However, it is also common to perform only a pre-saccharification step of about 40 to 90 minutes at a temperature above 50° C., followed by a complete saccharification during fermentation in a process known as simultaneous saccharification and fermentation (SSF).

Prior to entering the fermentation tank, the slurry must be cooled to about ambient temperature. The slurry is typically pumped through a heat exchanger to cool the slurry. It is important that the slurry remain in a relatively fluid form during this process. As the slurry thickens due to cooling, it places added pressure on the heat exchanger. A process improvement that reduces this pressure on the heat exchanger is an advantage to the ethanol producer.

Fermentation can be performed using yeast, e.g., a Saccharomyces spp. After fermentation, ethanol is recovered by distillation. The residual solids and liquids can be dried to make the fermentation co-product dried distillers grains (DDG) and dried distillers grains and solubles (DDGS). A portion of the liquid streams from the distillation (referred to as backset or stillage) can be recycled back to the process.

Conventional dry grind ethanol plants typically use approximately 29 to 33% solids in the slurry tank; however, when the process of the invention is used, the percentage of solids in the slurry tank can be increased to approximately 35 to 60% solids. Thus, when using the method of the present invention, the total solids entering the slurry tank can be about 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or 60%.

The viscosity of the slurry throughout the ethanol production process is a further component of ethanol production. The continuous flow process for ethanol production requires that the slurry be low enough in viscosity to move through pumps and pipes at a continuous rate. A slurry that gets too viscous can plug pipes, overflow tanks and cause undue stress on pumping equipment. In addition, a slurry that is not viscous enough can also cause problems as the solids in the slurry can fall out of the slurry and build up in pipes and pumps which also can cause plugging problems or undue stress on equipment. In some embodiments, the viscosity can be less than about 4000 cP, 3500 cP, 3000 cP, 2500 cP, 2000 cP, 1500 cP, 1000 cP, 500 cP, 400 cP, 300 cP, 200 cP, 100 cP, or 50 cP. Viscosity will vary depending on where in the ethanol production process the viscosity is measured.

In some embodiments, a product produced from the methods of the inventions include, but not limited to, alcohol (e.g., ethanol, methanol, butanol, propanol), lactic acid, an amino acid, fructose, citric acid, propanediol, dried distiller grain, dried distiller grain and solubles. In some embodiments, the product is an oil, a protein, a fiber, or yeast. In representative embodiments, the product may be ethanol, butanol, or yeast.

In particular, dried distiller grain and dried distiller grain and solubles are economically important co-products of corn-to-ethanol production. Dried distiller grain and dried distiller grain and solubles are primarily used as animal feed. Recognized value attributes of dried distiller grain and solubles are: consistency, physical characteristics (e.g. flowability, color, odor), and composition (e.g. protein and fiber content). Improvements in dried distiller grain and solubles benefit ethanol producers, commodity marketers, and the animal production industry.

The present invention is directed to a novel process for producing a starch rich flow that provides for increase in the amount of biomass that is processed without the typical problems associated with greater biomass, for example, jamming the heat exchanges.

Accordingly, a first aspect of the invention provides a method of producing a biomass-derived product, comprising: filtering through at least one paddle screen a high solids liquefaction slurry (i.e., a high solids liquifact) that comprises starch and fiber, thereby separating the starch into a starch rich stream and the fiber into a fiber rich stream; and fermenting the starch rich stream to produce a biomass-derived product.

A second aspect of the invention provides a method of processing a high solids liquefaction slurry to produce a biomass-derived product, comprising: filtering through at least one paddle screen a high solids liquefaction slurry that comprises starch and fiber, thereby separating the starch into a starch rich stream and the fiber into a fiber rich stream; and fermenting the starch rich stream to produce a biomass-derived product.

The high solids liquefact may be produced using any method of biomass processing in which the biomass to be processed (ground plant material) is combined with a liquid, for example, in a slurry tank, where it is mixed and then moved to a liquefaction tank, where the slurry is subjected to liquefaction to produce a high solids liquefaction slurry or high solids slurry. The amount of plant material that can be used with the present invention will vary depending on the size of the processing plant, but in general, can be at least about 2% to about 30% greater (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30%) than the amount of biomass that is used in a conventional process for any given processing facility.

A “high solids liquefaction slurry” as used herein means a ground plant material that has been subjected to liquefaction having a high solids content.

As understood by those of skill in the art, the operating temperature and enzyme dosages during liquefaction for producing the high solids liquefact are adjusted to conform to the equipment that is in use in the plant.

In some embodiments, a high solids liquefaction slurry may comprise about 35% to about 60% solids (e.g., about 35, 36, 37, 38, 39, 40, 41, 42, 43, 44, 45, 46, 47, 48, 49, 50, 51, 52, 53, 54, 55, 56, 57, 58, 59, 60 percent solids, and any range or value therein). In some embodiments, a high solids liquefaction slurry may comprise about 50% to about 80% of starch (e.g., about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 percent starch, and any range or value therein) and about 5% to about 25% of fiber (e.g., about 5, 6, 7, 8, 9, 10, 11, 12, 13, 14, 15, 16, 17, 18, 19, 20, 21, 22, 23, 24, 25 percent fiber, and any range or value therein).

In some embodiments, a starch rich stream may comprise about 55% to about 95% of starch (e.g., about 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 percent starch, and any range or value therein). In some embodiments, a fiber rich stream may comprise about 5% to about 35% of fiber (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 percent fiber, and any range or value therein).

The flow rate of the high solids liquefaction slurry into the paddle screen can vary substantially depending on the capacity of the particular bioprocessing plant. Thus, in some embodiments, the flow rate of a high solids liquefaction slurry into a paddle screen can be about 100 gal/min to about 2500 gal/min (e.g., 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, or any range or value therein).

Use of a high solids liquefaction slurry allows processing of greater quantities of plant material, resulting in increased quantities of starch and fiber for further processing into, for example, biofuels. However, the increased solids and starch may not be handled efficiently by the standard bioprocessing equipment. For example, the higher levels of solids can block heat exchangers that are used to reduce the temperature of the stream as it moves through the production system. As a consequence, there is a need to continually clean the heat exchangers, resulting in a substantial reduction in efficiency. The present invention overcomes this problem by introducing at least one paddle screen (e.g., 1, 2, 3, 4, 5, 6, 7, 8, 9, 10, 11, 12, 13, 14, 15, 16, 17, 18, 19, 20 or more paddle screens) between the liquefaction and fermentation steps, which captures the solids (e.g., fibers; fiber rich stream) that are too large to pass through the screen and separates them from the liquefied starch (starch rich stream) that moves through the screen. These solids are scraped off the screen by the paddles and moved into a fiber rich stream, while the liquefied starch (starch rich stream) passes through the screen and into a tank for further processing (e.g., holding/catch tank and/or fermentation tank).

Paddle screens are known in the art and include but are not limited to those made available by Fluid-Quip, Inc. of Springfield, Ohio (See, U.S. Pat. No. 8,778,433). A paddle screen useful with this invention can include screen openings (a mesh size) of between about 50 μM to about 120004 (e.g., about 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200 μM, and any range or value therein). In some embodiments, the mesh size of the paddle screen can be about 50 μM to about 150 μM. In some embodiments, the mesh size of the screen can be 50 μM or less, 100 μM or less, 150 μM or less. Typical fiber size in a slurry can be about 200 μM to about 2000 μM. Those of ordinary skill in the art will recognize how to determine the size of the openings to achieve the desired filtration based on the knowledge of the size of the particles to be captured (e.g., fiber, solids) versus the size of the components (e.g., starch) that are to pass through.

Any number of paddle screens may be used with this invention. In some embodiments, invention comprises at least two paddle screens, optionally two to five paddle screens, two to ten paddle screens, one to twenty paddle screens, or two to twenty paddle screens. In some embodiments, each of the at least two paddle screens can have similar or different mesh sizes. Thus, in some embodiments, the at least two paddle screens each have a different mesh size. In some embodiments, the invention comprises two to five paddle screens, wherein at least two of the two paddle screens comprise a different mesh size. In some embodiments, the invention comprises about two to about ten paddle screens, or about two to about twenty paddle screens, wherein at least two, at least three, or at least four, of the paddle screens have a different mesh size from one another. Thus, for example, more than one paddle screen can be used wherein the different paddle screens have decreasing screen/mesh size (e.g., 500 μM, 250 μM, 100 μM and 50 μM), which can be used consecutively to filter the high solids liquefaction slurry. In a further example, the screen sizes of the more than one paddle screen can be 100 μM and 50 μM, or 1000 μM, 750 μM, 500 μM, 400 μM, 300 μM, 200 μM, 100 μM, 50 μM and 10 μM, and the like. Using the guidance provided herein, one of skill in the art of bioprocessing of plant material would be able to readily determine the appropriate number, screen size, and arrangement for the paddle screens for use with a high solids liquefaction slurry.

The starch rich stream may be moved directly into a fermentation tank for fermentation or may be first placed in a catch tank or holding tank for about one minute to about 8 hours (e.g., 1 min, 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.5 hours, 6 hours, 6.5 hours, 7 hours, 7.5 hours, 8 hours, and the like and any range or value therein) prior to fermentation.

In some embodiments, the starch rich stream may be subjected to liquefaction prior to fermentation. Thus, in some embodiments, the starch rich stream may be directed to a holding tank, after which it is subjected to liquefaction and then fermentation. In some embodiments, the starch rich stream may be directed to a holding tank, followed by a liquefaction tank, and then a fermentation tank. In other embodiments, the starch rich stream may be directed from the holding tank directly to the fermentation tank or may be directed from the paddle screen to a liquefaction tank then a fermentation tank or only to a fermentation tank. The choice of process would be determined based on the amount of starch and the degree to which it is liquefied after passing through the paddle screen.

Typical dry grind ethanol process utilizes a continuous flow of mash from the initial mixing to form a mash until the mash enters the fermentation tanks. The mash flows through this front end of the process in a continuous manner meaning the mash moves at a pre-determined flow rate from the mixer to the heat exchanger. Typical dry grind ethanol plants use this front end continuous flow process in conjunction with batch style fermentation.

In batch style fermentation, fermentation tanks are filled on a sequential basis and fermentation is performed without the continuous flow of the mash. Once a fermentation tank is considered to be complete, the contents of the fermentation tank are transferred to a beer well and the continuous flow process begins again with the contents of the beer well continuously flowing to a distiller to start the process of collecting ethanol. Ethanol and whole stillage are collected from the distillation process and the whole stillage is further processed by passing through a centrifuge to separate solids and liquids. The solids are collected and form the dried distillers grains and the liquid, referred to as thin stillage, is either recycled into the process to form mash or is concentrated further to form a syrup.

The rate of flow of the mash from the mixer through to the fermentation tanks is typically the same rate of flow as from the beer well through distillation. The rates of flow are linked in order to maximize the recycling of energy in the ethanol production process. For example, the heat exchanger removes heat from the mash just prior to the mash entering fermentation. The heat exchanger transfers this heat to water to generate steam which is used in the distillation process.

Accordingly, in some embodiments, the starch rich stream may have a flow rate from the paddle screen to a holding/catch tank or a fermentation or liquefaction tank of about 10 gal/min to about 1500 gal/min (e.g., 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, or any range or value therein). The at least one paddle screen may be used to control flow rate (gals/min).

Any type of fermentation process may be used and the ordinary skilled person would be able to determine the most appropriate fermentation for any given bioprocessing plant. In a conventional fermentation process, the fiber rich stream, which has been liquefied, is cooled and delivered to a fermenting tank. Enzymes are added to the fermenting tank, with glucoamylase being used to finish the conversion of the liquefied starch to glucose. Various other enzymes can be added to the fermenting tank to aid in, for example, the production efficiency of sugar, providing nutrients to the yeast, and viscosity reduction, etc. Yeast are also added to the fermenting tank which consume the sugars produced and create the fermentation products. The fiber rich stream can be diluted with liquid to bring the solids down. This step is used to ensure that the conventional fermentation is capable of fermenting the majority of the starch and sugar being delivered to it in the mash. Many slight variations exist on this process. For example, a process can add the glucoamylase, or other enzymes, to a tank that operates above fermentation temperature. This can be done to increase the efficiency of the enzymes by operating at a higher temperature.

Another exemplary fermentation incorporates the Cellerate™ system that uses the conventional starch to ethanol process as a long hold time fiber hydration step. The present invention increases the energy efficiency of fermentation through the Cellerate™ process or other fermentation/distillation processed by adding a starch rich stream from the paddle screen. This results in increased ethanol concentration, thereby increasing the efficiency of the fermentation/distillation system and provides the ability to process more grain and create additional ethanol.

Thus, in some embodiments, the starch rich stream, produced by passing the high solids liquefaction slurry through the paddle screen, is directed to a fermentation system (e.g., Cellerate™ fermentation system) without passing through the conventional side liquefaction heat exchangers, fermentation or distillation systems. Thus, in some embodiments, the starch rich stream, produced by passing the high solids liquefaction slurry through the paddle screen, is directed to a secondary fermentation system without passing through the conventional side liquefaction heat exchangers, fermentation or distillation systems. The secondary fermentation system is a separate set of fermenters used for the conversion of sugars to other products. In some embodiments, the starch rich stream also does not go through, for example, the Cellerate™ fiber pretreatment process, which would occur after conventional liquefaction. In some embodiments, the starch rich stream may be directed to one or more liquefaction tanks after the paddle screen and before fermentation, where the starch is subjected to liquefaction prior to fermentation.

The present invention further provides a fiber rich stream by filtering through at least one paddle screen a high solids liquefaction slurry that comprises starch and fiber, thereby separating the starch into a starch rich stream and the fiber into a fiber rich stream. In some embodiments, the fiber rich stream is subjected to liquefaction and/or fermentation. In some embodiments, prior to liquefaction and/or fermentation, the fiber rich stream may be directed to a catch tank or holding tank for about one minute to about 8 hours (e.g., 1 min, 2 min, 3 min, 4 min, 5 min, 6 min, 7 min, 8 min, 9 min, 10 min, 15 min, 20 min, 25 min, 30 min, 35 min, 40 min, 45 min, 50 min, 55 min, 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours, 5.5 hours, 6 hours, 6.5 hours, 7 hours, 7.5 hours, 8 hours, and the like and any range or value therein).

In some embodiments, a portion of the starch rich stream being held a catch/holding tank is recombined with a fiber rich stream (produced by passing a high solids liquefaction surly through a paddle screen) to produce a recombined fiber rich and starch rich stream and the recombined fiber rich stream and starch rich stream is subjected to a conventional liquefaction and/or fermentation process. In some embodiments, water (e.g., cook water) may be added to the recombined fiber rich stream and starch rich stream prior to or after liquefaction.

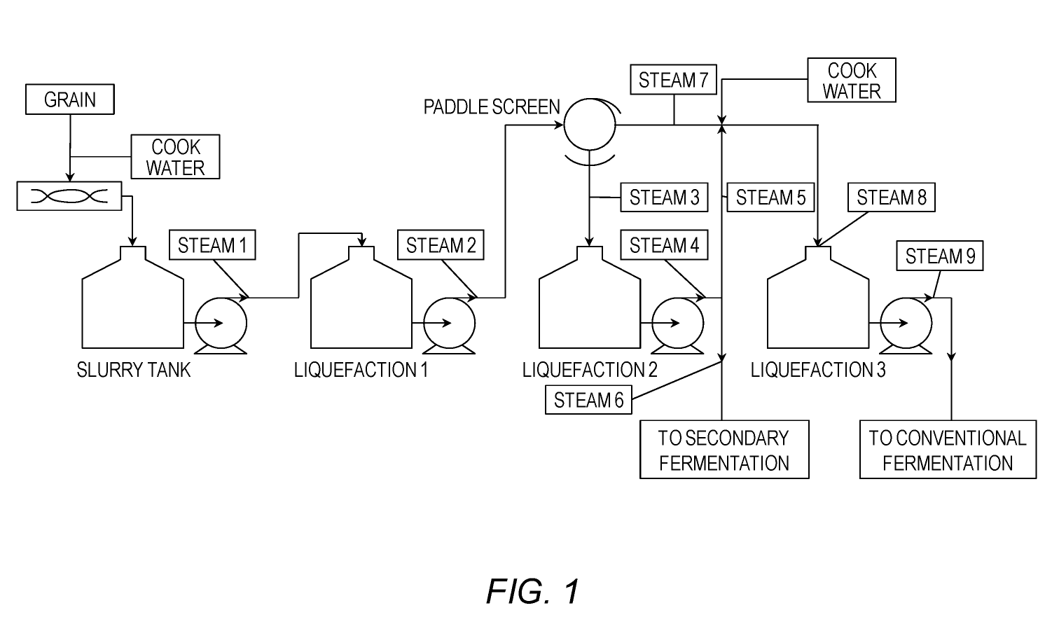

An exemplary system of this invention is provided in FIG. 1. This is meant to illustrate only one possible arrangement for carrying out the method of the invention. Many variations can be included that still fall within the presently claimed invention. Following is an outline of the process as set forth in FIG. 1.
1. Grain—any grain feedstock may be used in the production of dry grind ethanol, in representative embodiments, corn is the grain that is used as the plant material for producing the high solids liquefaction slurry
2. “Cook water” is generally a combination of evaporator condensate, CO2 scrubber water, fresh water, and thin stillage (backset). The choice and composition of this stream is envisioned as the much the same that which is used in a conventional dry-grind plant. However, in some embodiments, the percentage of backset that may be used at the two cook water insertion points can be less than is typically used in convention bioprocessing systems. Backset contains solids that are generally under 50 microns and typically very little starch; for this reason, using a smaller percentage of backset at the slurry tank and a greater percentage going into stream 6 of the present system will increase the percentage of starch coming through stream 3.
3. Stream 1—Here, the grain has been mixed with the cook water and allowed to soak in the slurry tank. Various temperatures and retention times can be used. The solids in the slurry tank can be at the level used in a conventional plant, approximately 30 to 36%. However, increasing the solids to higher levels increases the efficiency of a bioprocessing system. Stream 1 may be heated on its way to Liquefaction 1 tank, if the temperature in the slurry tank is not as high as desired. This heating can be accomplished in a variety of ways well known to those in the bioprocessing industry. The flow is allowed residence time in Liquefaction 1 tank to hydrolyze the starch and produce the high solids liquefaction slurry.
4. Stream 2 coming out of Liquefaction 1 tank comprises a solids level that is essentially the same as in Stream 1. If a heating method is used that injects steam, the solids in Stream 2 may be decreased by a small amount. Stream 2 is then sent to the paddle screen where it is split into two separate streams, Stream 3 and Stream 5.
5. Stream 3 is the centrate flow that has passed through the at least one paddle screen. Stream 3 contains dissolved solids and fine solids that are small enough to pass through the holes of the screen in use and is considered the “starch rich stream.” In this exemplary system, the starch rich stream, Stream 3, is directed to the Liquefaction 2 tank.
6. Stream 4—Stream 3 has been given further residence time in Liquefaction 2 tank. This residence time allows hydrolyzation of any unhydrolyzed starch passing through the screen. This stream may be then split in two directions (Stream 5 and Stream 6). A set flow (Stream 6) is sent to Secondary Fermentation and any additional flow may be routed as Stream 5 to Stream 7.
7. Stream 5—Stream 5 is used to control the level in Liquefaction 2 tank. The flow from the paddle screen may need modulation and therefore the paddle screen may be set to allow more flow than is required into Liquefaction 2 tank. The excess flow may then be pumped from the outlet of Liquefaction 2 tank to stream 7.
8. Stream 6—Flow of the starch-rich stream from Liquefaction 2 tank, Stream 4, is directed to Secondary Fermentation tank. This may be controlled by a flow control valve.
9. Stream 7 is the “cake” portion of the flow coming through the at least one paddle screen. This stream contains any solids too large to pass through the screen(s), as well as any hydrolyzed starch contained in the liquid and is termed the “fiber rich stream.” Additional cook water may be added at this point to dilute the solids to a level appropriate for fermentation in the conventional fermenters. The amount of added water is readily deter mined by those of skill in the bioprocessing based on the bioprocessing system in use.
10. Stream 8 includes Stream 7 after the additional cook water and any excess from Stream 4 (Stream 5) has been added. Stream 8 is directed to Liquefaction 3 tank where it is given additional residence time for starch hydrolyzation.
11. Stream 9—The contents of Liquefaction 3 tank are directed through Stream 9 to conventional fermentation.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1. Paddle Screen Separation of a Starch Rich Flow

This example describes the production and bioprocessing of a high solids starch rich stream

The initial slurry and separation is performed at high solids levels because it allows longer residence times in the liquefaction system and it reduces the gallons of water going into the Cellerate system, which increases fermentation times. The Cellerate system is an exemplary fermentation system that may be used with the method of the invention.

Water is added to the fiber rich conventional fermentation stream prior to fermentation to reduce the final ethanol concentrations to a manageable level in conventional bioprocessing systems. This is accomplished by adding water at a point after the fiber rich stream has been separated in the paddle screen (into a starch rich stream and a fiber rich stream). This addition may be at any point between the entrance into liquefaction and the exit of liquefaction. The later the dilution water is added the longer the liquefaction time may be.

The timing of the addition of the water is often determined by the amount of bacterial growth in the dilution water. If bacteria levels are high enough to cause issues in conventional fermentation, the water would be added earlier in the liquefaction process to allow more time at higher temperatures for disinfection.

The determination of liquid stream use at the two points of water addition also needs to be considered. If a significant portion of fine fiber is present in the backset, it must be determined how best to turn that portion into fermentable sugars. If a simple dosage of cellulosic enzymes is sufficient, then all of the backset should be added prior to the paddle screen. This sends a greater portion of this fine fiber straight to Cellerate fermentation. If the fine fiber requires the Cellerate pretreatment to liberate the sugars, then the backset should be used as the dilution water, which forces the fine fiber portion through the conventional system and the Cellerate pretreatment before the Cellerate fermentation.

Process Flows: Assuming that all of the backset would be used prior to the paddle screen.

Inflow to Paddle Screen: (FIG. 1, Stream 2)
307 gallons per minute (gpm), 50% solids, 70% of total solids as starch

Outflow to Secondary Fermentation: (FIG. 1, Stream 6)
42 gpm, 50% solids, 86% of total solids as starch

Outflow to Conventional Liquefaction: (FIG. 1, Stream 7)
265 gpm, 50% solids, 66% of total solids as starch

Outflow to Conventional Fermentation after Dilution Water Added (FIG. 1, Stream 9)
404 gpm, 35.2% solids, 66% of total solids as starch

TABLE 1
System with Flows

| | Slurry | Cellerate Liq | Conv Liq | |
|---|---|---|---|---|
| Corn | 1,680 | 229 | 1,451 | lbs/min |
| Ferm Solids | 1,092 | 186 | 907 | lbs/min |
| NonFerm Solids | 336 | 9 | 326 | lbs/min |
| Solids | 50% | 50% | 50% | |
| Water Flows | | | | |
| Condensate | 158 | 0 | 95 | gpm |
| Backset | 126 | 0 | 0 | gpm |
| Total Outflow | 307 | 42 | 404 | gpm |

The foregoing is illustrative of the present invention, and is not to be construed as limiting thereof. The invention is defined by the following claims, with equivalents of the claims to be included therein.

1. A method of producing a biomass-derived product, comprising:
filtering through at least one paddle screen a high solids liquefaction slurry that comprises starch and fiber, thereby separating the starch into a starch rich stream and the fiber into a fiber rich stream; and
fermenting the starch rich stream to produce a biomass-derived product.

2. A method of processing a high solids liquefaction slurry to produce a biomass-derived product, comprising:
filtering through at least one paddle screen a high solids liquefaction slurry that comprises starch and fiber, thereby separating the starch into a starch rich stream and the fiber into a fiber rich stream; and
fermenting the starch rich stream to produce a biomass-derived product.

3. The method of claim 1, wherein the high solids liquefaction slurry comprises about 35% to about 60% solids.

4. The method of claim 1, wherein the liquefaction slurry comprises about 50% to about 80% of starch and about 5% to about 25% of fiber.

5. The method of claim 1, wherein the starch rich stream comprises about 55% to about 95% of starch.

6. The method of claim 1, wherein the fiber rich stream comprises about 5% to about 35% of fiber.

7. The method of claim 1, comprising collecting the starch rich stream in a first catch tank or holding tank after the filtering through the at least one paddle and prior to fermenting.

8. The method of claim 1, wherein the starch rich stream is subjected to liquefaction prior to fermentation.

9. The method of claim 1, comprising subjecting the fiber rich stream to liquefaction and/or fermentation.

10. The method of claim 1, comprising collecting the fiber rich stream in a second catch tank or holding tank prior to liquefaction and/or fermentation.

11. The method of claim 1, wherein the at least one paddle screen comprises a screen having a mesh size between about 50 μM to about 1200 μM.

12. The method of claim 1, wherein the at least one paddle screen comprises a single paddle screen having a mesh size between about 50 μM to about 150 μM; optionally about 100 μM.

13. The method of claim 1, wherein the at least one paddle screen comprises at least two paddle screens each having a different mesh size, optionally 2 to 5 paddle screens, 2 to 10 paddle screens, or 2 to 20 paddle screens.

14. The method of claim 1, wherein the starch rich stream has a flow rate into the holding tank or to a fermentation or liquefaction tank of about 10 gal/min to about 1500 gal/min.

15. The method of claim 7, further comprising:
combining a portion of the starch rich stream from the holding tank with the fiber rich stream to produce a recombined fiber rich and starch rich stream and
subjecting the recombined fiber rich and starch rich stream to a conventional liquefaction and/or fermentation process.

16. The method of claim 15, wherein water is added to the recombined fiber rich stream and starch rich stream prior to or after liquefaction.

17. The method of claim 1, wherein the biomass-derived product is ethanol, butanol, or yeast.
